(19) United States
(12) Patent Application Publication
(10) Pub. No.: US 2024/0341860 A1
(43) Pub. Date: Oct. 17, 2024

(54) SYSTEM AND METHOD FOR ILLUSTRATING A POSE OF AN OBJECT

(71) Applicant: Medtronic Navigation, Inc., Lafayette, CO (US)

(72) Inventors: Michael R. Leners, St. Francis, MN (US); Elliot C. Schmidt, Minneapolis, MN (US); Brett D. Jackson, Shoreview, MN (US)

(21) Appl. No.: 18/630,474

(22) Filed: Apr. 9, 2024

Related U.S. Application Data
(60) Provisional application No. 63/495,951, filed on Apr. 13, 2023.

(51) Int. Cl. A61B 34/20; A61B 34/10; A61B 90/00; G06F 3/04817; G06T 7/00; G06T 17/00

(52) U.S. Cl. CPC A61B 34/20; A61B 34/10; A61B 90/361; A61B 90/37; G06F 3/04817; G06T 7/0012; G06T 17/00; A61B 2034/105; A61B 2034/2055; A61B 2090/378; G06T 2207/30048

(57) ABSTRACT

A system and method to provide output regarding an imaging system. The output includes, in various embodiments, a display illustrating portions features and portions selected by a user. The imaging system may include a user moveable and/or holdable imaging system.

### CROSS-REFERENCE TO RELATED APPLICATIONS

This application claims the benefit of and priority to U.S. Provisional Patent Application 63/495,951 filed on Apr. 13, 2023. The subject application includes subject matter related to the subject matter disclosed in U.S. Provisional Patent Applications 63/420,274 filed on Oct. 28, 2022; 63/420,315 filed on Oct. 28, 2022; and 63/420,328 filed on Oct. 28, 2022, all of which are incorporated herein by reference.

### FIELD

The present disclosure relates to acquisition of image data of a subject, and particularly to acquisition and display of image data collected from a tracked imaging system.

### BACKGROUND

This section provides background information related to the present disclosure which is not necessarily prior art.

An imaging system can be used to image various portions of a subject. The subject can include a patient, such as a human patient. The portions selected to be imaged can be internal portions that are covered by skin or other tissue. A location of imaged portions of the subject may be selected to be known within the imaging system. The locations can be defined or established relative to instruments placed in the subject (e.g., a location of a heart wall relative to a catheter) or a location of the imaged portion relative to the instrument acquiring the image data.

An imaging modality may be used to generate image data that are used to render images of an interior of the subject. For example, x-rays may be used to generate image data of an interior of a subject. The images may be displayed for viewing by a user.

### SUMMARY

This section provides a general summary of the disclosure, and is not a comprehensive disclosure of its full scope or all of its features.

The subject disclosure relates to illustrating a position, including a pose of an instrument relative to a patient. The patient may be a human or non-human patient. Further, the patient or subject may be a non-living object where the location of an instrument within the subject is selected to be tracked.

A representation of the patient may be fit or morphed to illustrate or represent a size of the patient. Therefore, relative portions of the patient, such as relative portions of the anatomy, are determined or known relative to one another based upon the patient that is the current patient during a procedure. An avatar or indicia of the patient may be generated or altered to a specific patient substantially in real time during a procedure.

An instrument can be tracked relative to the patient. A representation of the instrument may be superimposed on the avatar of the patient. The avatar of the patient may provide a visual illustration of various portions of the anatomy of the patient without requiring image data being acquired of the patient. The display being viewed by the user may be generated without requiring image data of the patient, such as fluoroscopy image data.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments, not all possible implementations, and are not intended to limit the scope of the present disclosure.

FIG. 1 is an environmental view of an exemplary imaging and navigation system;

FIG. 2A is a screen shot of a representation of an instrument at a tracked pose relative to an avatar of a subject, the screen shot including an image taken at a first orientation;

FIG. 2B is similar to FIG. 2A, but with the image at a second orientation that is about 90° relative to the first orientation;

FIG. 3 is a schematic view of a patient in an operating theater;

FIG. 4 is a schematic view of a user and a patient in an operating theater, according to various embodiments;

FIG. 5 is a detailed view of an instrument being moved relative to a patient;

FIG. 6 is a screen shot of a display illustrating a position of an instrument relative to an avatar;

FIG. 7 is a flowchart of a method, according to various embodiments;

FIG. 8 is a schematic illustration of an atlas and an image;

FIG. 9 is an illustration of images in two planes of a heart, according to various embodiments including ultrasound;

FIGS. 10A and 10B illustrate movement of a tracked instrument over time;

FIG. 11 is a flowchart illustrating a method of showing movement of a tracked instrument over time, according to various embodiments;

FIG. 12A illustrates a tracked pose of an instrument relative to an image in a plane in two orientations, according to various embodiments;

FIG. 12B illustrates a tracked pose of an instrument relative to an image in a plane in two orientations, according to various embodiments;

FIG. 13 illustrates an exemplary screen shot with right and left panels, on the right is an avatar of a subject with a housing of an imaging device and a field of view of the imaging device overlaid on the avatar, on the left is an image captured by the imaging device;

FIG. 14 illustrates an exemplary screen shot with right, left, and center panels, the center panel includes an avatar of a subject with a housing of an imaging device overlaid on the avatar, a first field of view in the center panel corresponds to an image captured by the imaging device shown in the left panel and a second field of view in the center panel corresponds to an image captured by the imaging device shown in the right panel;

FIG. 15 illustrates an exemplary screen shot with four panels, each panel shows a different orientation of a heart icon representing a heart being scanned with an imaging device, areas of the heart that have been scanned are displayed with different shading, coloring, etc. to distinguish from areas of the heart that have not yet been scanned;

FIG. 16 illustrates an exemplary screen shot with a sensor icon and a range icon overlaid on the avatar to identify a position and range of a sensor; and

FIG. 17 illustrates an exemplary screen shot with a left panel including a first respiration sensor icon and a second respiration sensor icon arranged on the avatar, and a right panel including a graphical representation of movement between first and second respiration sensors on a subject's body.

Corresponding reference numerals indicate corresponding parts throughout the several views of the drawings.

### DETAILED DESCRIPTION

Example embodiments will now be described more fully with reference to the accompanying drawings.

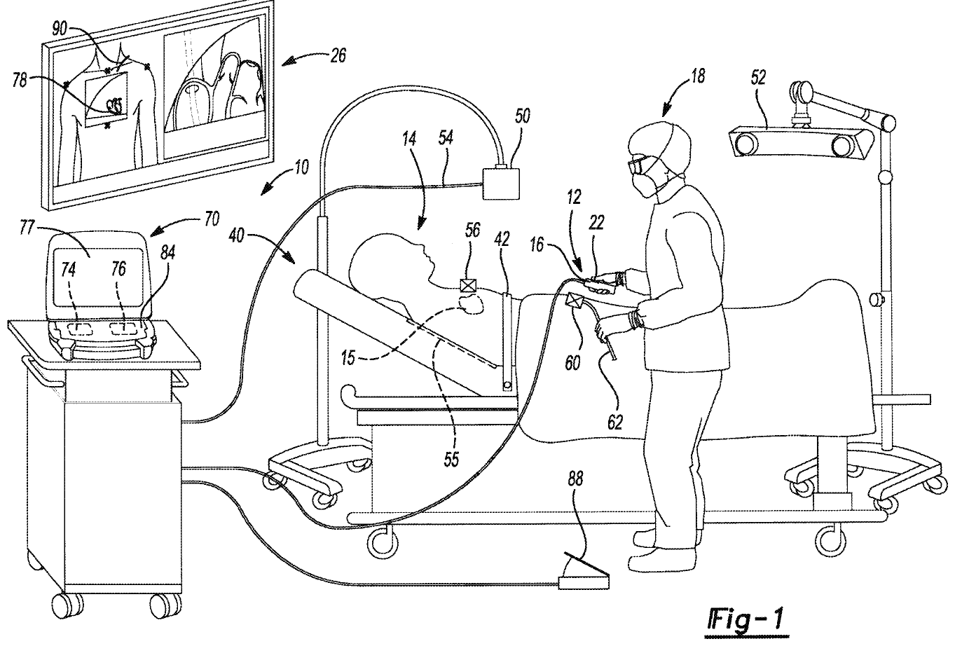

FIG. 1 is a diagram illustrating an overview of a navigation system 10 that can be used for various procedures. The navigation system 10 can be used to track the location of an item, such as an implant or an instrument, and at least one imaging system 12 relative to a subject, such as a subject 14 that may include a human patient and/or other living or non-living subject. It should be noted that the navigation system 10 may be used to navigate any type of instrument, implant, or delivery system, including: catheters, stylets, leads for cardiac rhythm management devices such as pacemakers, leadless pacemakers and delivery systems therefor, guide wires, arthroscopic systems, ablation instruments, stents, orthopedic implants, spinal implants, deep brain stimulation (DBS) probes, mechanical parts, Transesophageal Echocardiography (TEE), intra-cardiac echocardiography (ICE), etc. Non-human or non-surgical procedures may also use the navigation system 10 to track a non-surgical or non-human intervention of the instrument or imaging device. Moreover, the instruments may be used to navigate or map any region of the body. The navigation system 10 and the various tracked items may be used in any appropriate procedure, such as one that is generally minimally invasive or an open procedure.

The navigation system 10 can interface with or integrally include an imaging system 12 that is used to acquire pre-operative, intra-operative, or post-operative, or real-time image data of the patient 14. For example, the imaging system 12 can be an ultrasound imaging system (as discussed further herein) that has a tracking device 22 attached thereto. The imaging system 12 may also include any suitable CT system, x-ray system, C-arm, O-arm® imaging system, magnetic resonance imaging system, etc. The tracking device 22 may be tracked with the tracking system to determine a pose of the imaging system 12. The pose may include an orientation (e.g., three or more degrees of orientation (e.g., yaw, pitch, and roll) and/or a position (e.g., three degrees of freedom in physical space (e.g., x-axis, y-axis, and z-axis). The tracking system 10 may further determine appropriate pose information regarding the tracking device 22. The pose of the imaging system 12 can then be determined based on the tracked pose of the tracking device 22. The imaging system 12 may be used to generate image data to provide images for viewing with a selected display device 26, which may any appropriate display device including an augmented and/or virtual reality display device worn/used by the user 18 such as those disclosed in U.S. Pat. App. Pub. No. US2018/0078316A1, incorporated herein by reference.

It will be understood any appropriate subject can be imaged and any appropriate procedure may be performed relative to the subject. The navigation system 10 can be used to track various tracking devices, as discussed herein, to determine locations of the patient 14. The tracked poses of the patient 14 can be used to determine or select images for display to be used with the navigation system 10. The initial discussion, however, is directed to the navigation system 10 and the exemplary imaging system 12.

In the example shown, the imaging system 12 includes an ultrasound (US) imaging system with an US housing 16 that is held by a user 18 while collecting image data of the subject 14. It will be understood, however, that the US housing 16 can also be held by a stand or robotic system while collecting image data. The US housing 16 and included transducer can be any appropriate US imaging system 12, such as the M-TURBO® sold by SonoSite, Inc. having a place of business at Bothell, Washington. Associated with, such as attached directly to or molded into, the US housing 16 or the US transducer housed within the housing 16 is at least one imaging system tracking device 22. The tracking device 22 may be any appropriate tracking device such as an electromagnetic (EM) tracking device and/or an optical tracking device. The tracking devices can be used together (e.g., to provide redundant tracking information) or separately. Also, only one of the two tracking devices may be present. It will also be understood that various other tracking devices can be associated with the US housing 16, as discussed herein, including acoustic, ultrasound, radar, electrical impedance, and other tracking devices. Also, the tracking device 22 can include linkages or a robotic portion that can determine a location relative to a reference frame. Any suitable tracking device of any suitable tracking system may be used, such as, tracking devices and systems as understood by one skilled in the art.

In various embodiments, a secondary imaging system may also be presented and/or used to generated image data of the patient 14. The second imaging system may include an O-arm® imaging device sold by Medtronic Navigation, Inc. having a place of business in Louisville, Colorado, USA, a C-arm imaging system, or other appropriate imaging system. The second imaging system can include those disclosed in U.S. Pat. Nos. 7,188,998; 7,108,421; 7,106,825; 7,001,045; and 6,940,941; all of which are incorporated herein by reference. The second imaging system, however, need not be present and the imaging system 12, which may be only the US transducer, may be the only imaging system to generate images to be displayed. As discussed herein, various other image data may also be displayed such as an avatar 120 of the patient 14.

The imaging system 12 may be tracked, as discussed above. Various tracking systems may include and/or require calibration of the imaging system. Thus, the pose of the tracking device 22 relative to a plane of the US imaging system may be determined and/or known. Various systems and methods are disclosed in U.S. Pat. Nos. 6,379,302; 6,669,635; 6,968,224; 7,085,400; 7,831,082; 8,320,653; 8,811,662; and 9,138,204 all of which are incorporated herein by reference.

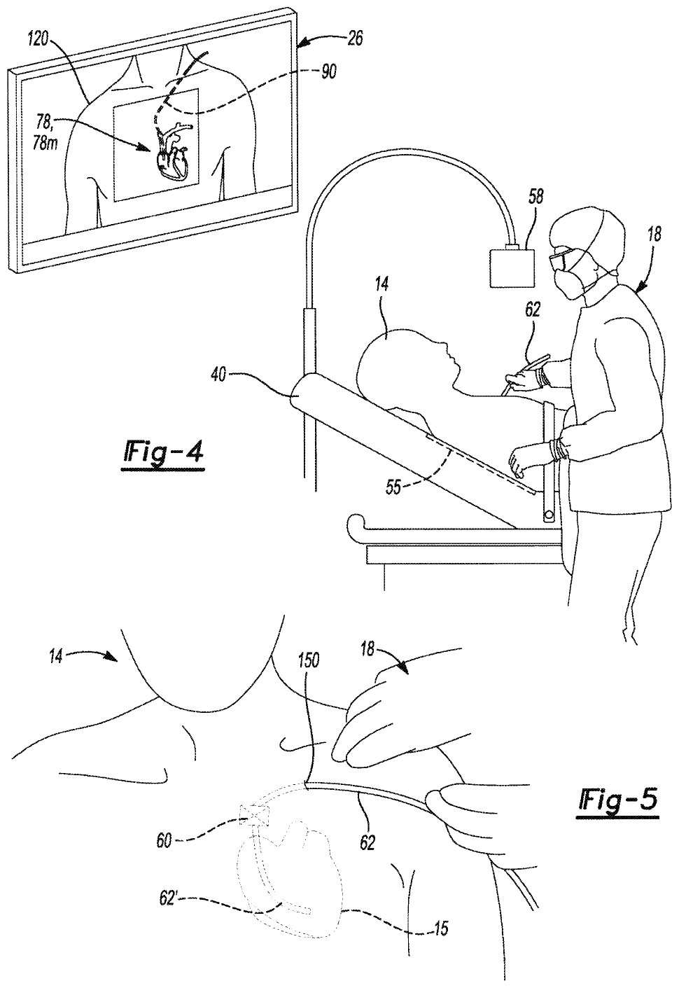

As illustrated in FIGS. 1, 3, and 4, the patient 14 can be fixed in a selected pose relative to a selected object, such as onto an operating table 40, but is not required to be fixed to the table 40. The table 40 can include a plurality of straps 42 or other fixation devices. The straps 42 can be secured around the patient 14 to fix the patient 14 relative to the table 40. Various apparatuses may be used to position the patient 40 in a static position on the operating table 40. Examples of such patient positioning devices are set forth in commonly assigned U.S. patent application Ser. No. 10/405,068, published as U.S. Pat. App. Pub. No. 2004/0199072 on Oct. 7, 2004, entitled “An Integrated Electromagnetic Navigation And Patient Positioning Device”, filed Apr. 1, 2003 which is hereby incorporated by reference. Other known apparatuses may include a Mayfield® clamp.

The navigation system 10 includes at least one tracking system. The tracking system can include at least one localizer. In one example, the tracking system can include an electromagnetic (EM) localizer 50. The tracking system can be used to track instruments relative to the patient 14 or within a navigation space. The navigation system 10 can use image data from the imaging system 12 and information from the tracking system to illustrate locations of the tracked instruments, as discussed herein. The tracking system can also include a plurality of types of tracking systems including an optical localizer 52 in addition to and/or in place of the EM localizer 50. When the EM localizer 50 is used, the EM localizer can communicate with or through a localizer communication 54 that may be wired or wireless. The EM localizer 50 may also include and/or take the form of an alternative pad or flat EM localizer 55.

The optical localizer 52 and the EM localizer 50 can be used together to track multiple instruments or used together to redundantly track the same instrument. Various tracking devices, including those discussed further herein, can be tracked and the information can be used by the navigation system 10 to allow for an output system to output, such as a display device to display, a position of an item. Briefly, tracking devices can include a patient or reference tracking device 56, also known as a dynamic reference frame, to track the patient 14, an instrument tracking device 60 to track an instrument 62, and/or other appropriate tracking devices for one or more portions. Patient or reference tracking device 56 and instrument tracking device 60 may include those disclosed in U.S. Pat. Nos. 8,060,185 and 8,644,907, both incorporated herein by reference. The tracking devices allow selected portions of the operating theater to be tracked relative to one another with the appropriate tracking system, including the optical localizer 52 and/or the EM localizer 50. The reference tracking device 56 can also or alternatively be positioned on an instrument and positioned within the patient 14, such as within a heart 15 of the patient 14.

It will be understood that any of the tracking devices 22, 56, 60 can be optical or EM tracking devices, or both, depending upon the tracking localizer used to track the respective tracking devices. It will be further understood that any appropriate tracking system can be used with the navigation system 10. Alternative tracking systems can include radar tracking systems, acoustic tracking systems, ultrasound tracking systems, electrical impedance tracking systems, radio frequency beacon, and the like. Exemplary tracking systems include those disclosed in U.S. Pat. Nos. 7,676,268; 8,532,734; and 8,494,608, all incorporated herein by reference. Each of the different tracking systems can be respective different tracking devices and localizers operable with the respective tracking modalities. Also, the different tracking modalities can be used simultaneously as long as they do not interfere with each other (e.g., an opaque member blocks a camera view of the optical localizer 52).

An exemplary EM tracking system can include the STEALTHSTATION® AXIEM™ Navigation System, sold by Medtronic Navigation, Inc. having a place of business in Louisville, Colorado. Exemplary tracking systems are also disclosed in U.S. U.S. Pat. No. 7,751,865, issued Jul. 6, 2010 and entitled “METHOD AND APPARATUS FOR SURGICAL NAVIGATION”; U.S. Pat. No. 5,913,820, titled “Position Location System,” issued Jun. 22, 1999 and U.S. Pat. No. 5,592,939, titled “Method and System for Navigating a Catheter Probe,” issued Jan. 14, 1997, all herein incorporated by reference.

Further, for EM tracking systems it may be necessary to provide shielding or distortion compensation systems to shield or compensate for distortions in the EM field generated by the EM localizer 50. Exemplary shielding systems include those in U.S. Pat. No. 7,797,032, issued on Sep. 14, 2010 and U.S. Pat. No. 6,747,539, issued on Jun. 8, 2004; distortion compensation systems can include those disclosed in U.S. patent Ser. No. 10/649,214, filed on Jan. 9, 2004, published as U.S. Pat. App. Pub. No. 2004/0116803, all of which are incorporated herein by reference.

With an EM tracking system, the EM localizer 50 and the various tracking devices can communicate through an EM controller. The EM controller can include various amplifiers, filters, electrical isolation, and other systems. The EM controller can also control the coils of the EM localizer 50 to either emit or receive an EM field for tracking. A wireless communications channel, however, such as that disclosed in U.S. Pat. No. 6,474,341, entitled “Surgical Communication Power System,” issued Nov. 5, 2002, herein incorporated by reference, can be used as opposed to being coupled directly to the EM controller. The EM controller can be incorporated into a navigation processing system 70.

It will be understood that the tracking system may also be or include any appropriate tracking system, including a STEALTHSTATION® TRIA®, TREON®, and/or S7™ Navigation System having an optical localizer, similar to the optical localizer 52, sold by Medtronic Navigation, Inc. having a place of business in Louisville, Colorado. Further, alternative tracking systems are disclosed in U.S. Pat. No. 5,983,126, to Wittkampf et al. titled “Catheter Location System and Method,” issued Nov. 9, 1999, which is hereby incorporated by reference. Other tracking systems include an acoustic, radiation, radar, electrical impedance, etc. tracking or navigation systems.

The navigation system 10 can include a navigation processing unit or module 74 that can communicate or include a navigation memory 76, which may be included in the navigation processing system 70. The navigation processing system 70 may further include a display device 77. The navigation processing unit 74 can include a processor and memory (e.g., microprocessor, a central processing unit, etc.). In various embodiments, the navigation processing unit may execute instructions to determine one or more poses of the tracking devices based on signals from the tracking devices. The navigation processing unit 74 can receive information, including image data, from the imaging system 12 and tracking information from the tracking systems, including the respective tracking devices and/or the localizers 50, 54. Image data can be displayed as an image 78 on the display device 26. The display device may be separate from and/or integrated into the navigation system, thus, the display device 26 may include the display device 77. The navigation system 70 can include appropriate input devices, such as a keyboard 84. It will be understood that other appropriate input devices can be included, such as a mouse, a foot pedal 88 or the like which can be used separately or in combination. Also, all of the disclosed processing units or systems can be a single processor module (e.g., a single central processing chip) that can execute different instructions to perform different tasks.

An image processing unit or module can process image data from the imaging system 12 and a separate first image processor (not illustrated) can be provided to process or pre-process image data from the imaging system 12. The image data from the image processor can then be transmitted to the navigation processor 74. It will be understood, however, that the imaging systems need not perform any image processing and the image data can be transmitted directly to the navigation processing unit 74. Accordingly, the navigation system 10 may include or operate with a single or multiple processing centers or units that can access single or multiple memory systems based upon system design.

In various embodiments, the imaging system 12 can generate image data that may be used to compose the image 78 and define an image space that can be registered to a patient space or navigation space that is defined by and/or relative to the patient 14. In various embodiments, the position of the patient 14 relative to the imaging system 12 can be determined by the navigation system 10 with the patient tracking device 56 and the imaging system tracking device(s) 22 to assist in and/or maintain registration. Accordingly, the position of the patient 14 relative to the imaging system 12 can be determined.

Manual or automatic registration of the image space to the subject space can occur. In various embodiments, the registration can occur by matching fiducial points in image data with fiducial points on the patient 14. Registration of image space to patient space allows for the generation of a translation map between the patient space and the image space. According to various embodiments, registration can occur by determining points that are substantially identical in the image space and the patient space. The identical points can include anatomical fiducial points or implanted fiducial points. Exemplary registration techniques are disclosed in U.S. patent application Ser. No. 12/400,273, filed on Mar. 9, 2009, now published as U.S. Pat. App. Pub. No. 2010/0228117 and in U.S. Pat. No. 9,737,235, issued Aug. 22, 2017, both incorporated herein by reference.

According to various embodiments, the imaging system 12 can be used with an un-navigated or navigated procedure. In a navigated procedure, a localizer and/or digitizer, including either or both of an optical localizer 52 and/or an electromagnetic localizer 50, 55 can be used to generate a field and/or receive and/or send a signal within a navigation domain relative to the patient 14. The navigated space or navigational domain relative to the patient 14 can be registered to the image 78. Correlation, as understood in the art, is to allow registration of a navigation space defined within the navigational domain and an image space defined by the image 78. The patient tracker or dynamic reference frame 56 can be connected to the patient 14 to allow for a dynamic registration and maintenance of registration of the patient 14 to the image 78.

Once registered, the navigation system 10 with or including the imaging system 12, can be used to perform selected procedures. Selected procedures can use the image data generated or acquired with the imaging system 12. Further, the imaging system 12 can be used to acquire image data at different times relative to a procedure. As discussed herein, image data can be acquired of the patient 14 prior to the procedure for collection of automatically registered image data or cine loop image data. Also, the imaging system 12 can be used to acquire images for confirmation of a portion of the procedure. Thus, image data may be acquired at any appropriate time and may be registered to the patient 14.

Registration and navigated procedures are discussed in U.S. Pat. No. 8,238,631, incorporated herein by reference. Upon registration and tracking of the instrument 62, a graphic representation 90 (e.g., an icon, indicium, animation or other or visual representation) may be displayed relative to, including overlaid (e.g., superimposed) on, the image 78. The image 78 may be an appropriate image and may include one or more 2D images, such as 2D images that are acquired at different planes. Images may also be a 3D image, or any appropriate image as discussed herein.

In addition to registering the subject space to the image space, however, the imaging plane of the US imaging system 12 can also be determined. By registering the image plane of the US imaging system 12, imaged portions can be located within the patient 14. For example, when the image plane is calibrated to the tracking device(s) 22 associated with the US housing 16 then a position of an imaged portion of the heart 15, or other imaged portion, can also be tracked as disclosed in U.S. Pat. Nos. 6,379,302; 6,669,635; 6,968,224; 7,085,400; 7,831,082; 8,320,653; 8,811,662; and 9,138,204 all of which are incorporated herein by reference.

Once the patient 14 is in condition for a procedure, the patient 14 may be positioned on the table 40, as noted above. The display 26 and/or the display 77 may display various information regarding the patient 14 and/or other information selected by the user 18. As noted above, the display 26 may illustrate the image 78 that may be acquired with the imaging system 12. The image 78 may also be displayed on the display 77. Also as noted above, the instrument 62 may be tracked and the graphic representation 90 of the instrument 62 may be displayed on the display 26, such as relative to the image 78. In addition and/or alternatively, the graphic representation 90 may also be displayed relative to a patient avatar 120.

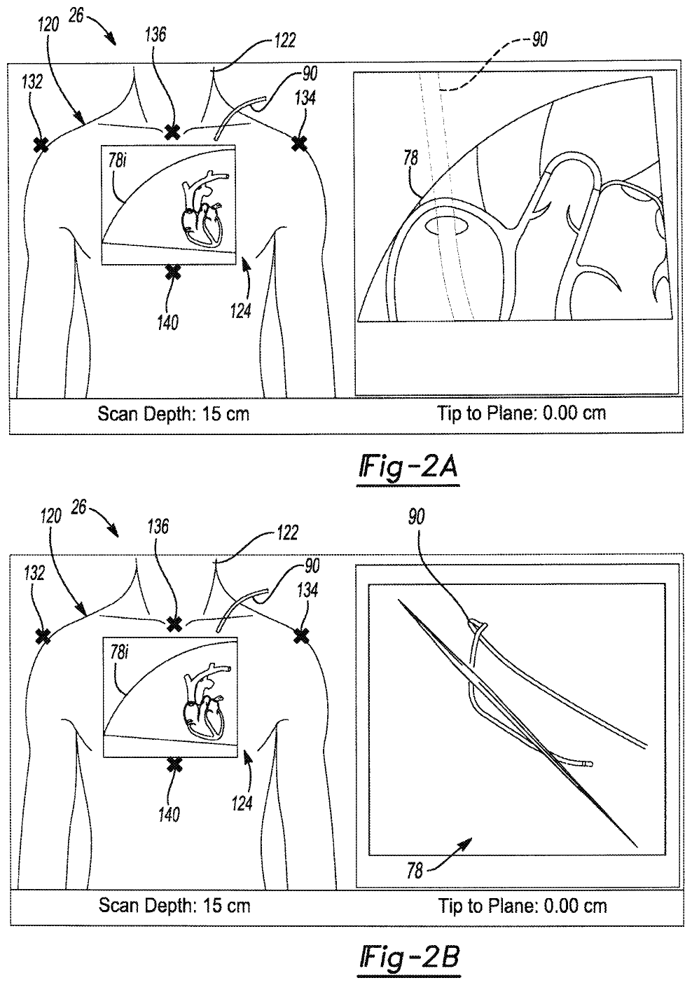

The patient avatar 120 may be illustrated on a selected display, as illustrated in FIGS. 2A and 2B, such as the display 26. The patient avatar 120 may be based on a general avatar that includes various features of the patient 14, as noted herein, but is sized to the current and specific patient. The avatar 120 may be illustrated as a two-dimensional (2D) image and/or a three-dimensional (3D) image. The avatar 120 may be any appropriate shape to assist in identifying various positions and relative positions of portions of the patient 14. The avatar 120, according to various embodiments, is sized to the specific patient as disclosed herein.

The patient avatar 120 may generally illustrate a portion of the patient 14 and may be sized relative to the patient 14. For example, the patient avatar may illustrate an inferior portion of a neck 122, a pectoral region 124, and other appropriate regions of the patient 14. The patient avatar 120 may be displayed on the display device 26 to illustrate a general representation of the patient 14 without requiring an image acquisition of the same portion of the patient 14. Therefore, the patient avatar 120 may provide a general roadmap or indication of the position of various tracked portions, as discussed further herein. The avatar 120 may however, as discussed herein, be morphed to match a relative size of the patient 14 to provide an indication of relative anatomical portions of the patient 14 and a pose of the tracked portions, such as the instrument 62, relative thereto.

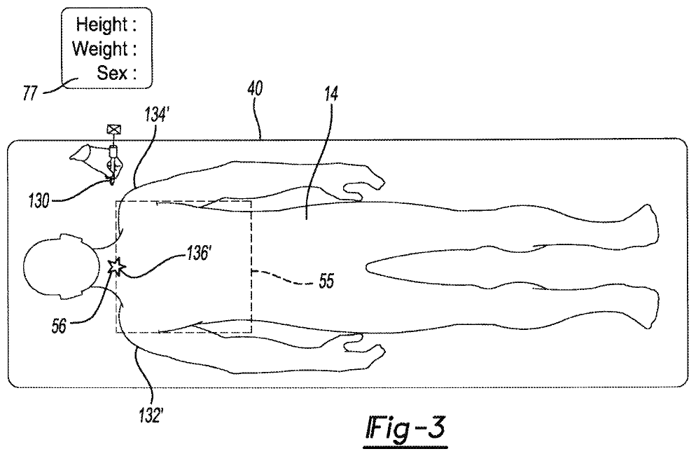

With continuing reference to FIG. 2 and additional reference to FIG. 3, the avatar 120 may be generally sized to the patient 14 according to one or more appropriate procedures. The avatar 120 may be sized or morphed in real time to match relative dimensions of the patient 14. The relative positions of the anatomic portions may include a relative position of a suprasternal notch and an exterior shoulder or inferior jugular vein, or any other suitable anatomical location(s).

In various embodiments, for example, the user 18 may provide or include information regarding the patient 14. As noted above, the navigation processing system 70 may include various inputs that allow the user 18 to input information. Therefore, as illustrated in FIG. 3, the user 18 may input information such as a height, a weight, and a sex of the patient 14. The input information is used by the processor module 74 to execute selected instructions to size the patient avatar 120 to the actual patient 14. The resizing of the avatar 120 may be based upon recalling of a selected or known predetermined size from a database, reconfiguring or resizing the patient avatar 120 based upon the input information, or other appropriate information. Therefore, the avatar 120 may be sized to substantially match the patient 14 without acquiring an image of the patient 14 for display on the display device 26.

In addition or alternatively thereto, the user 18 may also identify or size the avatar 120 to the patient 14 with a tracked indicator or tracked member 130. For example, the tracked indicator 130, which may be an indicator or probe usable to touch a surface of the patient 14, may be moved by the user 18 to various predetermined positions on the patient 14 as indicated on the avatar 120. The tracked indicator 130 may be tracked with any appropriate tracking system, including those discussed herein according to various embodiments such as the localizer system of U.S. Pat. No. 5,983,126, hereby incorporated by reference. The user 18 may move the tracked indicator 130 to various points, such as the exterior shoulder portions 132′, 134′ as illustrated by the icons 132 and 134. Additionally, the user 18 may move the tracked indicator 130 to other portions such as a suprasternal notch 136 and a sternum 140 of the patient 14 or outer boundary positions. It is understood that the tracked indicator 130 may be moved to any appropriate position and the above-noted positions are merely exemplary. Nevertheless, based upon the tracked position of the tracked indicator 130, the avatar 120 may be sized to morph the identified points to a representation of the patient 14 for display on the display device 26. Also, the points on the avatar 120 may be registered to the patient 14 by identifying them in the patient space relative to the patient 14 and identifying them on the avatar 120.

The avatar 120 may also be morphed to actual measurements taken of the patient 14. For example, a distance between the shoulder points 132′, 134′ on that the patient 14 may be measured, such as with a tape measure or other measuring device. The measured distance may be input within the navigation processing system at 77. The avatar 120 may be sized based upon the measurements.

Regardless of the technique, the avatar 120 may be matched or morphed to substantially mimic the shape of the patient 14. The avatar 120, as discussed further herein, may then be used as an illustration of a pose of the tracked instrument, such as the instrument 62, during a procedure on the patient 14. Thus, the avatar 120 may provide a representation of a position of the tracked instrument 62 without requiring an image acquisition of the patient 14, such as with a fluoroscopic imaging system. The avatar 120, as it is morphed to the specific patient may or will be displayed as a patient specific avatar. Thus, the avatar 120 may include general information, but is displayed and registered to the patient 14 as a patient specific avatar 120 including relevant locations and relative positions of various portions of the patient 14. The specific patient avatar illustrates the relative positions of the anatomy of the patient 14 that is the current patient. For example, the position of the heart 15 relative to the suprasternal notch.

The avatar 120 may be registered to the patient 14 in any appropriate manner, including those discussed above. In various embodiments, for example, when the tracked indicator 130 is tracked to determine positions of the patient 14 for marking or changing the avatar 120. The patient 14 after the various points are determined in both the patient 14 and the avatar 120 may be registered thereto. Additionally, and/or alternatively, the patient tracker 56 may be positioned on the patient 14 at an appropriate position. The position of the patient tracker 56 may be at the suprasternal notch 136′ which relates to a point on the avatar 120, such as the suprasternal notch 136. The position of the suprasternal notch 136 in the avatar 120 may be determined or known to be the position of the patient tracker 56 to allow for registration of the avatar 120 relative to the patient 14. Also, the patient tracker 56 may maintain registration even when the patient 14 moves relative to the localizer(s). It is understood that any appropriate number of the patient trackers 56 may be utilized, including each may be used for registration and/or more than one of the patient trackers 56 may be used.

The localizers, including the EM localizer 58, may also or alternatively be fixed or positioned relative to the patient 14 in an appropriate manner. For example, the localizer 58 may be fixed relative to the table 14 such that it is immovable during the procedure. Thus, the field generated with the EM localizer 58 can be at a known position relative to the patient 14 and allow for a registration of the avatar 120 to the patient 14 based upon the known position of the EM localizer 58 relative to the patient 14. The localizer 55 may also be positioned under the patient 14, such as between the patient 14 and the bed 40. Localizer 55 may include field generating portions, such as coils, that generate a field relative to the patient 14. The flat or panel localizer 55 may be positioned at a known position relative to the patient 14, such as aligning the shoulder edges 132′, 134′ to known or predetermined portions of the panel localizer 55. As the panel localizer 55 is at a known position relative to the patient 14, and the avatar 120 is morphed to the patient and 14, the avatar 120 may be registered to the patient 14 due to the position of the panel localizer 55.

Regardless, the avatar 120 may be registered to the patient 14. Registration of the avatar 120 relative to the patient 14 allows the avatar 120 to be used to assist navigation and guiding of the instrument 62, or any appropriate instrument relative to the patient 14. As the tracked position of the instrument 62 is determined with the navigation system 10, the pose of the instrument 62 may be illustrated with a graphical representation, such as the graphic representation 90 relative to the image 78 and/or the avatar 120.

As illustrated in FIGS. 2-5, the instrument's graphic representation 90 is displayed superimposed on the avatar 120. The user 18 may view the display 26 and understand the tracked position of the instrument 62 relative to the patient 14 by viewing the graphic representation 90 of the instrument 62 in the avatar 120 on display 26. The user 18 may then understand the relative position of the instrument 62 to the patient 14 and determine that the instrument 62 is generally moving and/or is positioned in a selected or procedure planned position. The user 18, thus, may understand this by viewing the display 26 and the avatar 120 without requiring additional or any image data being acquired of the patient 14.

In various embodiments, for example, image data may be acquired of the patient 14. As noted above, the imaging system 12 may acquire image data of the patient 14. The imaging system 12 may generally be a non-ionizing imaging system, such as an ultrasound imaging system. The ultrasound image may generally be collected at a single plane relative to the housing 16. The US imaging system 12 with the tracker 22 may be tracked relative to the patient 14 so that the image may also be registered to the patient 14. Thus, the image 78 may also be displayed superimposed on the avatar 120, such as the image superimposition 78i. The image may also be displayed separately from the avatar, as also illustrated in FIGS. 2A and 2B. FIG. 2A illustrates the image 78 at a first orientation. FIG. 2B illustrates the image 78 at a second orientation that is about 90 relative to the first orientation. The operator of the imaging system 12 may toggle between views to select the view that they find most suitable without moving the housing 16.

As the image 78 may be registered to the patient 14, a single image acquisition may occur and the image 78 may be displayed for the procedure. Further, as the image 78 is registered to the patient the graphic representation 90 of the instrument 62 may be displayed at the tracked pose on the image 78. Thus, the US imaging system 12 may be used at one point in time to acquire the image data, but it may be used to illustrate the selected portion of the patient 14, such as the heart, for an entire procedure. A constant imaging of the patient 14 is not required to illustrate or know a position of the instrument 62 as the instrument 62 is tracked and the image 78 is registered to the patient 14 due to the US tracker 22.

In a procedure, once the avatar 120 and/or the image 78 are registered to the patient, the user 18 may perform the procedure on the patient 14 with the tracked instrument(s). The procedure may be any appropriate procedure, such as placing a pacing lead and/or a catheter to assist in placing the lead. Other appropriate procedures may also be performed.

In various embodiments, the user 18 may form an incision, such as an incision 150 on the patient 14, and move the instrument 62 into the patient 14 through the incision 150. The tracked portion 60 on the instrument 62 may be tracked so that a position of the instrument 62 may be displayed relative to the avatar 120 and/or the image 78, as noted above. The instrument 62 may then be moved relative to the patient 14. In various embodiments, for example, the instrument 62 may be a catheter and/or lead for an implant, or a stylet or a delivery system for an implant such as a leadless pacemaker or a stent. As is understood by one skilled in the art, an implant, including an electrode, may be positioned relative to one or more portions of the heart 15 of the patient 14.

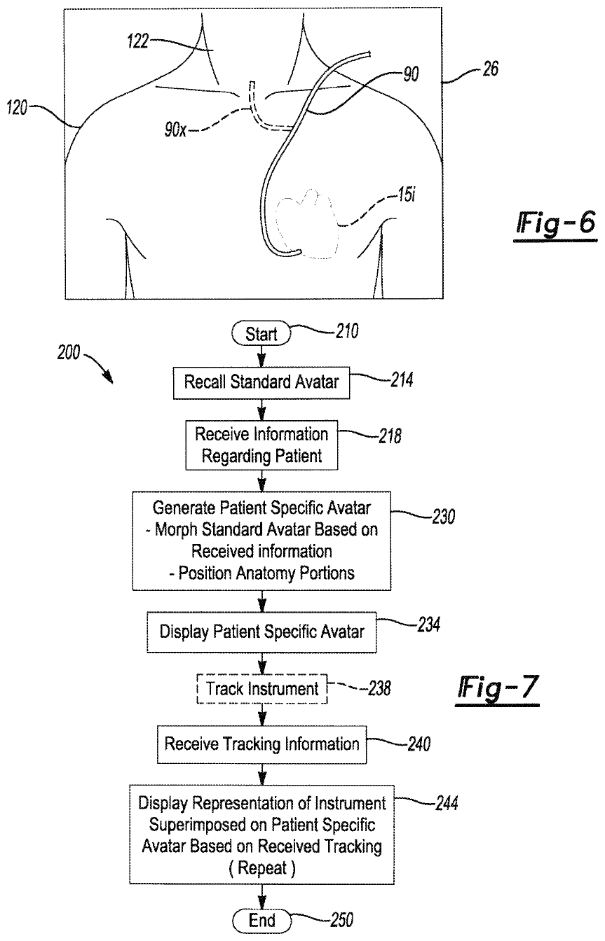

With continuing reference to FIG. 5 and additional reference to FIG. 6, the position of the instrument 62 may be illustrated with the graphic representation 90 over time and/or instantaneously relative to the avatar 120. The graphic representation 90 illustrates the pose of the instrument 62 tracked relative to the patient 14. The graphic representation 90 may be viewed by the user 18 to understand a real-time position of the instrument 62 within the patient 14 via the avatar 120. Again, the avatar 120 may be generated without requiring image data of the patient 14, such as with fluoroscopy.

The avatar 120 may also assist the user 18 in identifying or determining an orientation of the instrument 62 as being in a selected or in a not-selected pose relative to the patient 14. For example, as illustrated in FIG. 6, the avatar 120 may be viewed by the user 18 during a procedure. The user 18 may view the representation 90 of the instrument 62 as it moves in a selected direction and/or position. The tracked pose of the instrument 62 may be updated in substantially real time and displayed relative to the avatar 120. Therefore, the graphic representation 90 of the instrument 62 when it is moving in a not-selected position or and/or pose may also be illustrated and understood by the user 18. As illustrated in FIG. 6 a representation 90x may illustrate movement and/or pose toward a neck 122 of the patient 14. This may be a not-selected pose when the instrument 62 is selected to be moving toward the heart 15 of the patient 14. Therefore, the user 18 may view the graphic representation 90x and understand that the instrument 62 is moving away from the heart 15 of the patient 14. A representation of the heart 15 may also be displayed on the avatar 120 including the image 78 and/or with a heart representation 15i that is included in the avatar 120 and appropriately located with the patient specific information.

Thus, the user 18 may view and understand the position of the instrument 62 during the placement of an implant, which may include, for example, a stimulation lead for a selected system. The lead may include a pacing electrode be for a cardiac stimulation system, such as the AZURE®, ADAPTA®, or other appropriate heart pacemaker or defibrillation systems sold by Medtronic, Inc. having a place for business in Minnesota, USA.

Turning reference to FIG. 7, a method 200 for determining and displaying a patient specific avatar is illustrated. The process may begin in Block 210 and include recalling a standard avatar in Block 214. In recalling a standard avatar the processor, such as the processor 74, may recall a standard avatar including various anatomical features, including exterior dimensions of a patient's anatomy and selected internal anatomy such as a heart, physical location of the heart, jugular vein, or the like.

Information regarding the patient, such as the specific patient, may be received in Block 218. The information regarding the patient may be any appropriate information, including that discussed above. Therefore, the information may include general dimensions of the current patient, including a height, weight, sex, or the like. Information received in Block 218 may also include specific tracked points of predetermined portions (e.g., shoulder edges) of the patient 14. Further the received information may include measurements of the patient. Regardless, the received information of the patient may be patient specific information regarding the patient for a current or real-time procedure.

A patient specific avatar is then generated in Block 230. The generation of a patient specific avatar may include morphing or fitting the standard avatar based upon the received information. In morphing the standard avatar, the method may include altering or scaling dimensions of the standard avatar based upon a ratio given the information received regarding the current patient. The ratio may be based upon the known dimensions of the current patient based upon known or average dimensions of various anatomical portions relative to one another based upon the dimensions of the specific patient. This may also include having predetermined average positions and relative positions of various anatomical features. In addition and/or alternatively thereto, the tracked position of the instrument may identify specific anatomical locations. The standard avatar may then be morphed based upon the tracked specific locations for the specific patient. Thus, the avatar may be morphed based upon selected instructions including an algorithm to generate the patient specific avatar in Block 230.

After generating the patient specific avatar in Block 230, the patient specific avatar may be displayed in Block 234. As discussed above, the display 26 may display the patient specific avatar for selected procedures. The display of the patient specific avatar may allow the user 18 to identify and understand relative positions or locations of various portions of the current patient 14.

An instrument may then be tracked, optionally and according to various embodiments, in Block 238. Although the tracking of the instrument is an optional part of the method 200, such tracking may allow for information regarding a pose of an instrument to be determined. Regardless of whether the instrument is tracked, the method 200 may include receiving tracking information in Block 240.

Receiving tracking information in Block 240 may include tracking one or more tracking devices to determine a pose of various elements or portions, such as the pose of the instrument 62 via the instrument tracker 60 and/or the patient 14 via the patient tracker 56. The patient specific avatar may be registered to the patient based upon the known location of one or more specific portion(s) of the patient, such as the positioning of the patient tracker 56 in the suprasternal notch of the patient 14. The suprasternal notch of the avatar 120 may then be registered for determination of the pose of the instrument 62 relative to the patient tracker 56. Therefore, the pose of the instrument 62 may be displayed on the display device 26 such as the superimposed graphic representation 90 on the avatar 120.

Thus, the pose of the instrument may be superimposed on the avatar 120 in Block 244. Again, this allows a user 18 to view and understand a pose of the tracked instrument 62 on a representation of the patient 14 without requiring image data to be acquired to the patient 14. While image data of the patient 14 may be acquired, such as with the US imaging system 12, such image information is not required. The image data information from the US imaging system 12 may assist in finalizing or determining a specific location of the instrument 62 relative to a selected portion, such as a specific portion of the heart 15 of the patient 14. According, only a selected portion or area of the patient may be imaged. However, the position of the instrument 62 may be determined without the image data. Thus, the user may understand a position of the instrument 62, including a movement of the instrument 62 toward a selected position, such as the RV septum of the patient 14.

The method 200 may then end in Block 250. And ending the method 250 it is understood that the method 200 may be repeated as often as necessary or selected to allow for up to date and/or real-time tracking and illustration of a pose of the instrument 62 relative to the avatar 120. Thus, the method 200 may be repeated at a selected rate and for a selected period, such as selected by the user 18.

As noted above, moving the instrument 62 relative to the patient 14, such as relative to the heart 15, may be accomplished while tracking a pose of the instrument 62. As noted above, tracking a pose of the instrument 62 can involve attempting to determine a physical location and/or orientation of the instrument 62 in the physical space. The tracked posed may include a location which includes X, Y, and Z axis positions and orientation which may include yaw, pitch, and roll orientations in space. The physical space may also be referred to as navigation space and is generally defined relative to the patient 14 and/or the heart 15. The patient tracker 56 allows the pose of the patient 14, including the heart 15, to be tracked. Tracking a pose relative to a portion of the patient 14, including the heart 15, may include systems such as those disclosed in U.S. Pat. No. 7,697,972 issued Apr. 13, 2010; U.S. Pat. No. 8,046,052 issued Oct. 25, 2011; U.S. Pat. No. 8,180,428 issued May 15, 2012; and U.S. Pat. No. 8,663,120 issued Mar. 4, 2014, all of which are herein incorporated by reference.

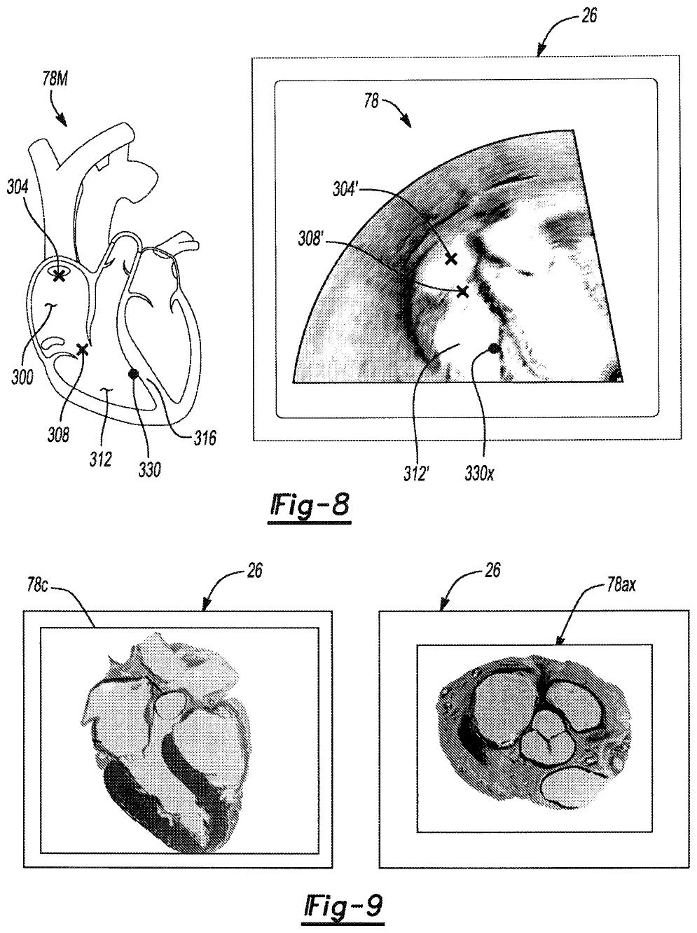

With continuing reference to FIGS. 1-7, and additional reference to FIG. 8, the instrument 62 may be tracked relative to the patient 14 which may be displayed such as within the graphic representation 90 relative to selected portions including the avatar 120, the image 78, and/or an atlas 78m, as illustrated in FIG. 8. The atlas 78m may be based upon the selected information, such as based on a plurality of images of patients that have been averaged and portions thereof identified. For example, the atlas 78m may be a model based on the image data from a plurality of images. The atlas 78m (also referred to as a model) may have selected portions therein that are identified. In various embodiments, the atlas 78m may include the following identified locations: a right ventricle 300, a superior vena cava ostium 304, a tricuspid valve 308, a right ventricle 312, and a right ventricle septum 316.

With reference to FIG. 8, the image 78 is an example of one image in a set of images that can be used to build the atlas 78m. The atlas 78m may also be generated using the patient's pre-procedure imaging like a CT or MRI. The image 78 may be obtained with a selected imaging system, including the US imaging system 12, as discussed above. A patient specific anatomy model may be generated using multiple 2D/3D views/planes stitched together, for example to reconstruct the endocardium of the heart. To facilitate building the atlas 78m, a clinician may identify various relevant portions or locations within the image 78. The user 18 may identify portion(s) or location(s) in the image 78 shown in the right-hand side of FIG. 8 as the superior vena cava entrance 304′, the tricuspid valve 308′, and/or the right ventricle 312′. Other locations may include endocardium or blood pool boundary may include the identification of the portions/locations within the heart. The location identification may be done manually by the user 18 and/or with assistance of an automatic location identification, such as segmenting a valve, etc.

Multiple images similar to the image 78, e.g., multiple similar images from a population of patients other than the patient 14, with similar identified portion(s) or location(s) may be combined to build the atlas 78m to illustrate the identified portion(s) or location(s) on a model of the imaged anatomy or heart, which model represents a typical or average patient anatomy. Alternatively, the atlas 78m may be built only from images of the anatomy of the patient 14, and the portion(s) or location(s) of interest applied to the atlas based on identification within the images of the patient's own anatomy, and/or based on a compilation of images from other patients' anatomy and an estimated and/or average position of the portion(s) or location(s) of interest within the compilation of images. The atlas 78m may also be morphed to actual measurements taken of the patient 14.

Based upon the user's 18 identification in the image 78 of the various portions/locations, which may include all of or selected number of portions/locations, a processing module or unit, such as the processor 74, may execute selected instructions to register or identify various portions in the image 78 and/or on the atlas 78m for viewing by the user 18. For example, in the atlas 78m the right ventricle septum may be identified as a selected point including a target 330. The image 78 may also, therefore, have the target identified thereon 330x to illustrate a proposed or selected position of the right ventricle septum (RV septum), e.g., to serve as a target for delivery of a catheter, stylet and/or pacing lead, or a leadless pacemaker. The predetermined portions in the model 78m, therefore, may be used to assist in identifying portions of the image 78 based on one or more portions identified by the user 18 and/or appropriate image identification systems. Therefore, the model 78m may be used to assist in identifying portions within the image 78 to assist in the procedure. The user may view the image 78 and identify or evaluate the target 330x prior to and/or during the procedure.

The image 78, as discussed above, may also include a plurality of images that are generated with the imaging system 12. The US imaging system 12 may acquire images of the patient 14, including the heart 15, in a plane. As the US housing 16 is moved, the plane of the image relative to the patient 14 may also change. Thus, the image 78 may include a plurality of images that are illustrated in one or more planes. For example, with reference to FIG. 9, schematic images that may represent an approximately coronal to the body co-ordinate system image 78c may be acquired and displayed and/or an image that is approximately axial to the body co-ordinate system 78ax may be displayed. The two images 78ap and 78ml may both be displayed and registered to the patient 14, as discussed above. Further, the tracked position of the instrument 62 may be displayed relative to one or both of the images 78ml and 78ap. Also, both of the images 78ml and 78ap may assist in registering or relating of the images to the model 78m. Additionally, the user 18 may view the images, as discussed above, and identify portions or locations thereon and of the model 78m that may be used to assist in identifying various other portions. For example, the user 18 may identify the tricuspid valve 308′ and the superior vena cava entrance 304′ and the model 78m may be used to identify various other portions or locations, such as the target 330x on the RV septum 330.

In addition to providing more than one view of the heart 15 of the patient 14, the two images 78ap and 78ml may also assist in registration to the model 78m. Thus, the identification of anatomy in the images 78 and/or one or both of the images and 78ap and 78ml, may be assisted by the registration of more than one image relative to the model or atlas 78m. As discussed above, the ultrasound imaging system 12 is registered to the patient 14, thus the images acquired with of the imaging system 12 may also be registered to the patient 14. The registration of the images to the patient may be based upon the tracking of the imaging system 12, such as with the imaging system tracking device 22 and the tracking of the patient 14, such as with the patient tracking device 56. Once the image 78 is registered to the patient 14, the instrument 62 may be tracked relative to the patient 14 and its pose may be displayed relative to the image 78. As discussed above, the pose of the instrument 62 may also be displayed relative to the avatar 120. The graphic representation 90 allows the user 18 to view the pose of the instrument 62 relative to both of the image 78 and the avatar 120.

Further, the instrument 62 may be tracked in real time. Therefore, the instrument may be displayed relative to the avatar 120 and/or the image 78 to provide a real time graphical representation of the instrument 62 relative to the heart 15, based upon a graphical representation displayed relative to the image 78. In addition to viewing the tracked pose of the instrument 62, the graphical representation may show the pose of the instrument 62 over time. Therefore, the graphic representation 90 may illustrate or provide a sense of an entire path of the instrument 62 as it moves from the incision 150 in the patient 14 to the current location within the patient 14. Further, the graphic representation 90 may provide a real-time feedback to the user 18 of a movement of the instrument 62.

In various embodiments, the images 78 may be acquired or based upon image data acquired at a selected time. For example, prior to the incision 150, image data may be acquired with the imaging system 12 to generate the image 78. Image 78 may thus be registered to the patient 14 and displayed with a display device 26 either alone and/or superimposed on the avatar 120. Nevertheless, the image 78 may not be a real time or current image and may be a static image. The instrument 62, however, may be tracked in real time and provide a current determination of the pose of the instrument 62. The graphic representation 90 may, therefore, be updated in real time and at a selected rate to illustrate a current position of the instrument 62 and/or a current shape or condition of the patient anatomy. The display of the graphical representation may also be updated in a selected rate to provide a perception of motion of the instrument 62. For example, a refresh of the pose of the instrument 62 and of the graphic representation 90 may be 10 times a second, 30 times a second, or any appropriate rate such as about one time a second to about 120 times per second. Thus, the pose of the instrument 62 may be illustrated to show a motion within the display device 26.

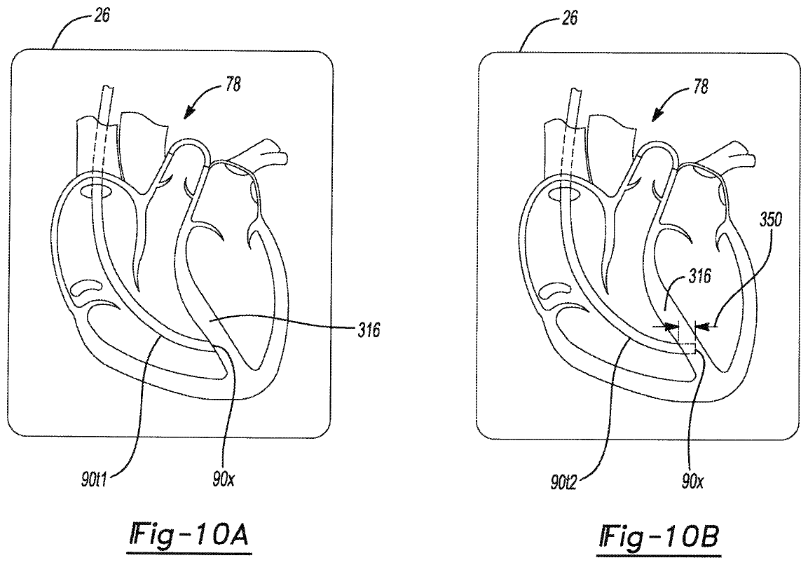

With reference to FIGS. 10A and 10B, the image 78 may be displayed on the display device 26. The image 78 may not change over time as the image 78 may be a static image that is acquired at a selected time, such as prior to positioning the instrument 62 within the patient 14. Nevertheless, the tracking device 60 may be tracked in real time on the device 62. Thus, as illustrated in FIG. 10A, the graphic representation 90 may include a first or initial time graphic representation 90t1. The graphic representation 90t1 may represent a first graphic representation at a selected time, that may be arbitrarily referred to as a first or initial time. After a selected period of time, a second graphic representation 90t2 may be displayed on the display device 26, as illustrated in FIG. 10B.

The graphic representation 90t2 may be displayed or have a selected position that is displaced a certain distance of 350 relative to the first graphic representation 90t1. The distance 350 may be any appropriate distance and may be based upon the various physical features, such as movement of a beating heart. The heart 15 of the patient 14 may beat (i.e., move due to the heartbeat), as is understood by one skilled in the art. Therefore, the graphic representation 90t2, including a terminal end 90x, may be shown in different positions based upon movements of the instrument 62. For example, as one skilled in the art will understand, when the terminal end of the instrument 62 contacts the RV septum 316, the terminal end of the instrument 62 may move due to the beating of the heart 15. Therefore, even if the user 18 is not moving the instrument 62, the instrument 62 may appear to move in real time. Real-time tracking of the instrument 62 with the tracking device and 60 may allow for a representation of the movement of the instrument 62 to be displayed on the display device 26. As discussed above, the movement of the instrument may be updated and displayed at any appropriate rate to provide a perception of movement to the user 18. Thus, the user 18 may be provided with additional information regarding a position of the instrument 62, such as being in contact with of the RV septum 316.

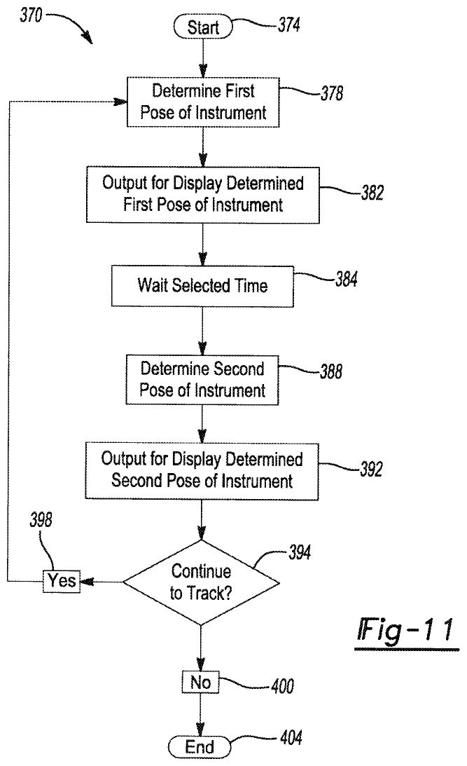

With reference to FIG. 11, a method or process 370 is illustrated. The process 370 may be a process that is executed by one or more of the processing units, such as processing unit 74. The method 370 may begin at start Block 374. After starting the process in Block 374, the process 370 may determine a first position or pose of the instrument in Block 378. Determining the first pose of the instrument may be performed with the navigation system, as discussed above. The pose of the instrument 62 may be known relative to the patient 14, or portions thereof such as the heart 15, in light of the tracking of the patient with the patient tracker 58.

After determining a pose of the instrument at a first time, an output for display of the determined pose of the instrument may be made in Block 382. The output of the determined pose may be based upon the registration of the image 78 and/or the avatar 120 to the physical space defined by the patient 14, as discussed above. Thus, the output may display the graphic representation 90 at a first position, for example of the first pose 90t1, as illustrated in FIG. 10A.

After determining and outputting the first pose, a wait or pause for a selected time may occur in Block 384. The wait for a selected time may be any appropriate time, such as about one second to about 120 seconds. The wait time may be any appropriate time to assist in illustrating a motion to the user 18, as discussed above. After the wait time, the second pose may be determined in Block 388. The second pose may be determined in the same manner as the first pose was determined in Block 384. An output for display of the second pose may be made in Block 392 to display the graphical representation of the instrument 62 at a second time, such as the graphic representation 90t2. As noted above, therefore, the pose of the instrument 62 may be displayed over a series of the events and/or time to show movement to the user 18.

A determination can be made to continue tracking at Block 394. If tracking is to be continued, a YES path 398 may be followed to again determine a first or subsequent pose in Block 378. The process 370 may provide an illustration to the user 18 for an appropriate period of time. If a tracking is not to be continued, a NO path 400 may be followed to end the process in Block 404. The process 370 may be used to illustrate and determine a pose of the instrument 62 over time for illustration relative to the image 78, as illustrated in FIGS. 10A and 10B.

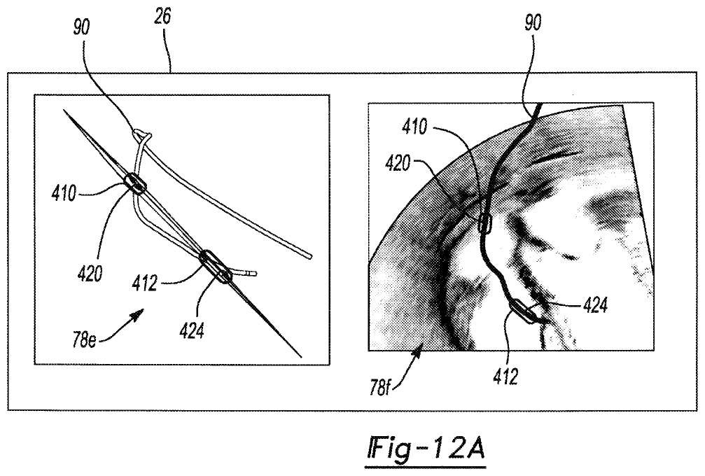

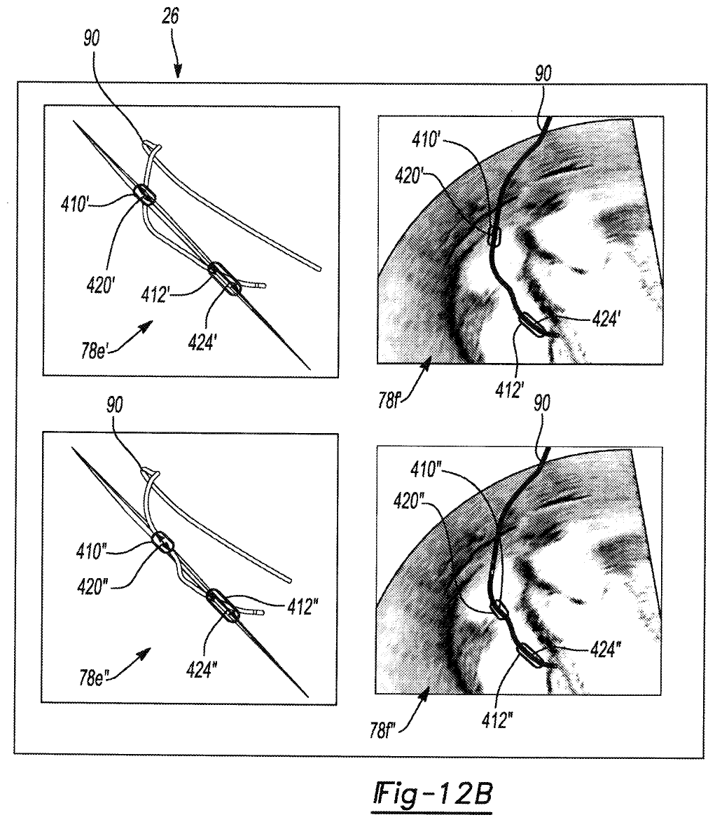

The display 26 may display the image of the patient 14 that is acquired with the imaging system 12. As discussed above, the image 78 may be an image that is acquired in a plane from the ultrasound housing 16. Therefore, rotation of the image may change a perspective of the image 78. With reference to FIG. 12A, the image may be viewed over the plane 78f or from an edge 78e. The edge image 78e may allow for a viewing and understanding of a perspective of the instrument 62, as illustrated by the graphic representation 90, relative to a plane of the image. In various embodiments, the image acquired with the imaging system 12 may be a static image that is acquired with the imaging system 12. It is understood, however, that a live or current image may also be acquired and displayed. Nevertheless, the position of the instrument 62 relative to the plane of the image may be illustrated by rotating the image, such as showing it from an edge as illustrated in the edge image 78e.

Further, the distance of the instrument 62 that is within the plane of the image may also be illustrated. For example, a first and second distance icon 410, 412 may be included or illustrated superimposed on both of the images 78e, 78f. The distance icons may be used to illustrate the distance that the instrument 62 is in the plane of the image. Thus, the user 18 may also understand that the portion of the instrument that is within the plane of the image and that other portions are not within the plane of the image.

The distance icon 410, 412 may be sized depending upon the length or portion of the instrument 62 that is within the plane of the image 78. Therefore, the first distance icon 410 may have a first distance or size 420. The second distance icon 412 may have a second distance or size 424. The two dimensions 420, 424 may be different or the same depending upon the amount of the instrument or length of the instrument that is within the plane of the image 78. If the instrument has a greater length within the plane, the distance icons 410, 412 may have greater dimensions. If any dimension of the instrument is smaller in the plane, then the dimension icons may be smaller. Regardless, the dimension icons 410, 412 may be displayed, such as superimposed on, the image 78 to represent the distance of the instrument within the image plane. This may allow the user 18 to understand or determine the distance of the instrument 62 within a plane of the imaging system and/or assist in moving of the imaging system to acquire a different perspective than in the image 78 relative to the instrument 62.

According to various embodiments, the display 26 may display the image of the patient 14 that is acquired with the imaging system 12 at more than one location. In other words, more than one image may be displayed for each position of the imaging system 12 at which image data is acquired. Moreover, one or more image data portions may be merged and/or used in a reconstruction of a displayed image. As discussed above and herein, all of the images and/or the reconstructed image may be registered to the patient 14 due at least to the tracking of the imaging system 12.

With reference to FIG. 12B, for example, the image 78 may be an image that is acquired in a plane from the ultrasound housing 16. Therefore, rotation of the image may change a perspective of the image 78. The image may be viewed as acquired at a first location (e.g., inferior from the suprasternal notch) as over a plane 78f′ or from an edge 78e′. These two views may be able to provide information to the user, as discussed above including location and/or dimension that the instrument graphic representations 90 passes through the plane 410′, 412′, 420′, 424′. Further, the image may be viewed as acquired at a second location (e.g., between selected ribs) as over a plane 78f″ or from an edge 78e″. These two views may be able to also provide information to the user, as discussed above including location and/or dimension that the instrument graphic representations 90 passes through the plane 410″, 412″, 420″, 424″. Given that the image portions 78e′, 78f′ are acquired at a different position than the image portions 78e″ 78f″, the graphic representations 90 is illustrated to pass through the planes at the different positions and/or dimensions 410′, 412′, 420′, 424′, 410″, 412″, 420″, 424″ and this information is displayed for the user. Further, it is understood that any appropriate number of location images may be acquired and/or displayed and two is merely exemplary.

Again, the edge image may allow for a viewing and understanding of a perspective of the instrument 62, as illustrated by the graphic representation 90, relative to a plane of the image. In various embodiments, the image acquired with the imaging system 12 may be a static image that is acquired with the imaging system 12. It is understood, however, that a live or current image may also be acquired and displayed. Nevertheless, the position of the instrument 62 relative to the plane of the image may be illustrated by rotating the image, such as showing it from an edge as illustrated in the edge image.

The instrument 62 may be tracked with any appropriate tracking system. As noted above, tracking and/or navigation systems include those disclosed above and those disclosed in U.S. Pat. No. 7,697,972 issued Apr. 13, 2010; U.S. Pat. No. 8,046,052 issued Oct. 25, 2011; U.S. Pat. No. 8,180,428 issued May 15, 2012; and U.S. Pat. No. 8,663,120 issued Mar. 4, 2014, all of which are herein incorporated by reference. The navigation system may allow for a determination of a pose of the instrument 62 in a physical location and/or orientation of the instrument 62 in the physical space. The tracked pose may include a location which includes X, Y, and Z axis positions and/or orientation which may include yaw, pitch, and roll orientations in space. A tracking and navigation system may also include one or more permanent magnets and/or magnetometers.

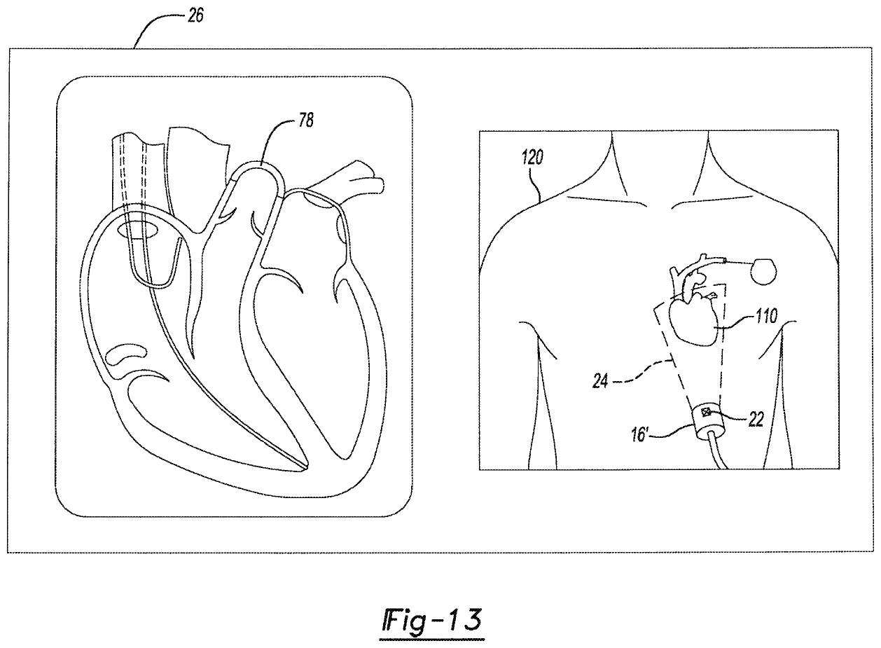

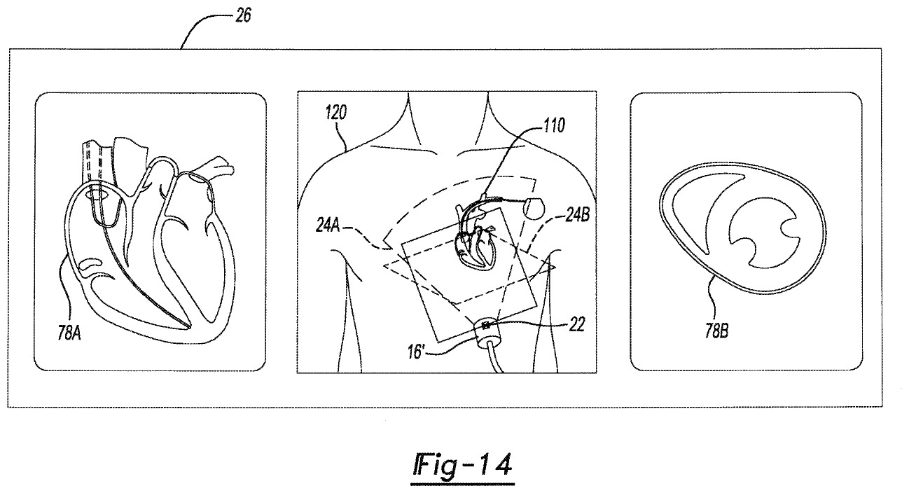

With reference to FIG. 13, the navigation processing system 70 is further configured to generate the display 26 of FIG. 13. The display 26 shows a probe housing icon 16′ of the housing 16 of the imaging system 12. That is, the icon 16′ is displayed relative to, such as superimposed on, the avatar 120. This allows the user to understand the pose of the probe 16 relative to the subject and the image data acquired with the probe 16.

Shown emanating from the probe housing icon 16′ is a field of view 24 of the imaging system 12. The portion of the anatomy within the field of view 24 captured by the imaging system 12 is shown as the ultrasound image 78. In the example illustrated, a heart image or model 110 is within the field of view 24. The heart image would be an actual ultrasound image captured by the imaging system 12. A 3D model would be a model created from segmented ultrasound images or from a registered CT or MRI scan. The location of the field of view 24 on the display 26 is determined based on the pose of the housing 16, as determined by tracking the position of the tracking device 22. Showing the field of view 24 on the display 26 assists the operator of the imaging system 12 with positioning the housing 16 to capture a desired portion of the anatomy. The user can see on the avatar 120 the portion of the anatomy within the field of view 24, which is displayed as the US image 78. If an area of interest is not within the field of view, the user can use the avatar 120 as a guide for maneuvering the housing 16 until the area of interest is within the field of view 24 and displayed in the ultrasound image 78. For example, the US image 78 of FIG. 13 is captured when the probe 16 is in the orientation of the probe housing icon 16′ shown in FIG. 13. If the area of interest is not visible in the US image 78 of FIG. 13, the user can use the avatar 120, the heart image or model 110, the housing icon 16′, and the field of view 24 as guides for maneuvering the actual housing 16 to bring the area of interest into the field of view 24. More specifically, if the area of interest is at the mid-papillary level of the heart, for example, the user can maneuver the actual housing 16 until the field of view 24 is at the mid-papillary level of the heart image or model 110. This is the case in FIG. 14, where the housing 16 has been maneuvered such that the field of view 24B extends through the mid-papillary level of the heart image or model 110, thereby resulting in the capture of US image 78B at the mid-papillary level of the heart. Thus, the user or selected processor executing instructions may view and understand the a current position of the probe and move the probe to a new position based on the current position.

The navigation processing system 70 can also be configured to generate the display 26 of FIG. 14. In the example illustrated, the display 26 includes three panels. The center panel shows the avatar 120 displaying the subject's heart, or other relevant anatomy, as the heart image or model 110. Also shown on the center panel is the probe housing icon 16′, a first field of view 24A, and a second field of view 24B. The first field of view 24A corresponds to the US image 78A on the left of the display 26. The second field of view 24B corresponds to the US image 78B on the right side of the display 26. The first field of view 24A and the second field of view 24B may be captured by two different housings 16, or by the same housing 16 at different times.

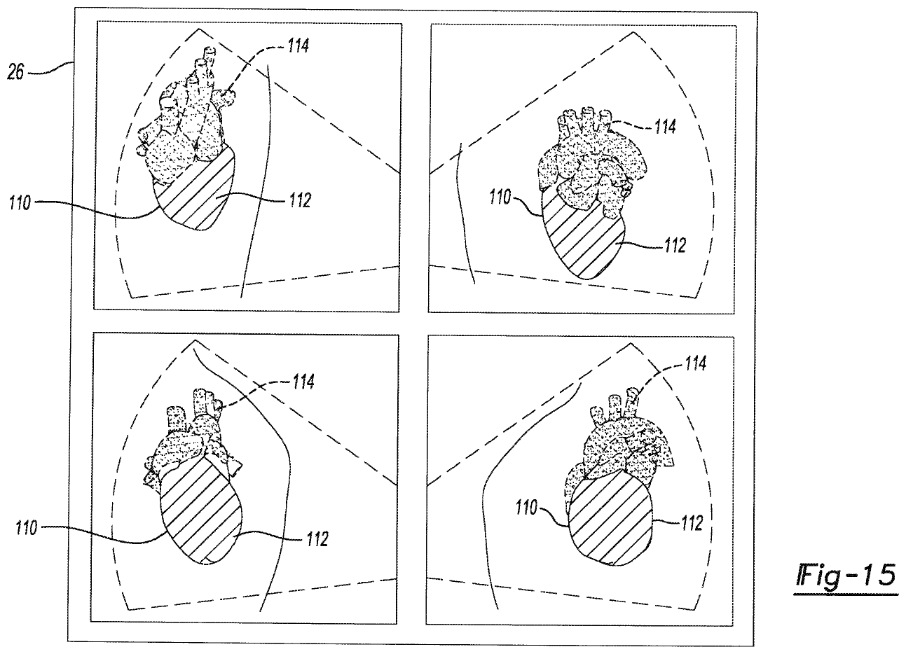

With renewed reference to FIG. 8 and additional reference to FIG. 15, the atlas model 78M may be generated by capturing a plurality of US images of the subject using the imaging system 12. The images collected of the subject, such as the heart, may also be used to morph an atlas of the heart of the current subject. In various embodiments, a user must thus maneuver the housing 16 to capture an entirety of the anatomy, such as an entirety of the heart. It is understood, however, that collecting a sufficient amount of image data of the subject may be appropriate to morph an atlas or generate an atlas.

For some users, it may be difficult to identify areas of the heart that have been captured and areas of the heart that have yet to be captured, whether to ensure a capture of an entirety of the heart or of all of the areas necessary to generate the appropriate atlas or model. The navigation processing system 70 may be configured to generate the display 26 of FIG. 15, which includes four panels displaying different sides of the heart image or model 110. The heart image or model 110 is displayed in any suitable manner that distinguishes areas of the heart that have been scanned by the imaging system 12 versus areas of the heart that have not been scanned by the imaging system 12. During the scanning process, the navigation system 70 tracks movement of the housing 16 relative to the anatomy. In the example illustrated, first portions 112 of the heart image or model 110, which are illustrated with solid lines, have already been imaged by the imaging system 12. The navigation processing system 70 tracks the position of the housing 16 relative to the anatomy, in this case the heart, to identify Second portions 114 of the heart image or model 110, which are illustrated in phantom, have yet to be imaged. The navigation processing system 70 is configured to update the display 26 as the heart is scanned to reflect the additional scanning. For example, and with respect to the images of FIG. 15, as the atriums of the heart are scanned the navigation processing system 70 is configured to update the heart image or model 110 to convert the atriums from second portions 114 in phantom to first portions 112.

Thus, during the scanning process, the navigation system 70 tracks movement of the housing 16 relative to the heart, or other portions of the anatomy being scanned, to identify portions of the heart that have been scanned versus portions that have not been scanned. The navigation system 70 continuously updates the images of FIG. 15 as described above to distinguish the portions of the heart that have been scanned versus the portions that have yet to be scanned based on tracked movement of the housing 16 relative to the heart.

The imaging system 12, including the ultrasound probe, may be moved relative to the subject to acquire an appropriate amount of image data, as discussed above. As illustrated in FIG. 15, the imaging and/or navigation processing system may be used to identify portions of the heart that have not been imaged or not imaged enough to generate a selected feature or type of image for display. For example, as discussed above, the image data acquired may be used to generate an image for analysis of the subject. A three-dimensional or volumetric image of a portion of the subject, such as the heart, may be generated based upon the image data acquired with of the imaging system 12. For example, the ultrasound probe generates data in a plane. Planes may intersect each other when generated or collected with the ultrasound probe. As the ultrasound probe is tracked during acquisition of the image data planes that may move and change relative to one another, such as being orthogonal. The image data acquired at different perspectives (e.g., different planes) that are known relative to one another allows a reconstruction to be made. The reconstruction may be of the heart as the image 110. The reconstruction may be a three-dimensional reconstruction based upon the image data collected with the ultrasound probe and the processor may illustrate portions of the heart needed to be imaged to reconstruct the 3D image, as noted above. Reconstruction techniques may include, but are not limited to, the following: computed tomography, specifically ultrasound computed tomography; and NeRF: Representing Scenes as Neural Radiance Fields for View Synthesis, Ben Mildenhall, Pratul P. Srinivasan, Matthew Tancik, Jonathan T. Barron, Ravi Ramamoorthi, Ren Ng (Aug. 3, 2020) (https://arxiv.org/abs/2003.08934), which is incorporated herein by reference in its entirety.

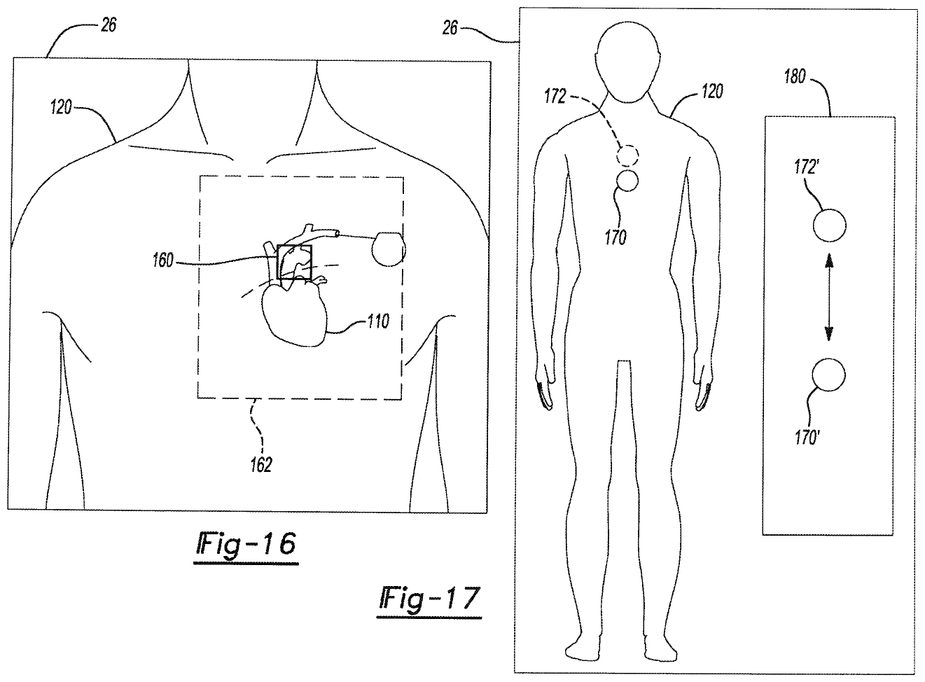

With reference to FIG. 16, the navigation processing system 70 is configured to generate a display 26 including a sensor icon 160 arranged relative to the avatar 120. The position of the sensor icon 160 represents the position of any of the localizers relative to the subject 14, such as any of the EM localizers 50, 55, or 58. Thus, a user may understand by viewing the avatar 120 the pose of the EM localizers 50, 55, or 58.

As discussed above and understood by one skilled in the art, the EM localizers 50, 55, or 58 may emit a field that induces a current in a tracking device, such as the tracking device 22. The display 26 also includes a range icon 162, which identifies the outer boundaries of the range of the particular localizer displayed. The range icon 162 identifies that extent (e.g., area or volume), in which the field emitted by the EM localizer is effective for navigation. Similarly, a field of view of an optical localizer may be displayed as the range icon 162. The range icon may be displayed relative to the subject aviator 120.

Displaying the sensor icon 160 and the range icon 162 facilitates positioning of the subject 14 and/or the localizer 50, 55, or 58 to capture an area of the body of interest. That is, the range icon 162 illustrates that extent to which the EM localizer 50, 55, or 58 is effective. As the avatar 120 illustrates the subject to be imaged and/or have tracking devices tracked relative thereto, the range illustrates the area in which a tracking device may be effectively tracked. Thus, the user may view the range icon 12 and decide if the volume included is effective for the procedure or not.

The localizer is registered in any suitable manner. For example, registration may be carried out with a reference tracker on the subject's body at a predefined location, such as the reference tracker 56. That is, the localizers 50, 55, or 58 may sense a reference marker placed on the subject at a known anatomical location and the localizer may be used to determine the pose of the localizer relative thereto. Further, the localizer range 162 may be relative to a second localizer that is tracked with a first localizer.

FIG. 17 illustrates another exemplary display 26 in accordance with the present disclosure. The display 26 of FIG. 17 displays the avatar 120 including a first respiratory sensor icon 170 and a second respiratory icon 172 relative to the avatar 120. The first respiratory icon 170 represents the position on the subject's chest of a first respiratory sensor including an EM tracker. The second respiratory icon 172 represents the position on the subject's back of a second respiratory sensor including an EM tracker. The trackers may be placed in the same general vicinity by convention. One region of high motion (e.g., substernal/subxiphoid region) and one region of lesser motion (upper back). Or alternatively the patch may be placed and the user selects from a generic menu of where it was placed from an array of options that would make sense (regions of the upper torso). It is typically desirable to arrange the first and the second respiratory sensors so that they are aligned with respect to the orientation of the avatar 120 in FIG. 17. The relative positions of the first and the second respiratory sensor icons 170, 172 in FIG. 17 allows medical personnel to determine whether the actual respiratory sensors are aligned. The display 26 also includes a respiration meter 180, which includes additional first respiratory icons 170′ and second respiratory icons 172′. During respiration, the icons 170′, 172′ move relative to each other to visually illustrate respiration. This allows monitoring for the phase and depth of respiration, identifying abnormalities with monitoring (e.g., tracker becomes disconnected or isn't well attached), and/or abnormalities in the regularity of the breathing of the patient, which may result in inconsistent tracking or imaging.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit that may also be referred to as a processor. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors or processor modules, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processor module” as used herein may refer to any of the foregoing structure or any other physical structure suitable for implementation of the described techniques. Also, the techniques could be fully implemented in one or more circuits or logic elements.

What is claimed is:

1. A system configured to assist a user with navigating an instrument relative to a subject, the system comprising:
an imaging system configured to image an anatomy, the imaging system including a probe maneuverable by the user;
a tracking system including a localizer, a probe tracker configured to be mounted to the probe, and a subject tracker configured to be mounted to the subject;
a display device including a display screen; and
a processor configured to:
generate an avatar of the subject and display the avatar in a first area of the display screen;
display an anatomy icon of the anatomy on the avatar in the first area of the display screen;
display a probe icon and a field of view icon of the probe in the first area of the display screen at a position relative to the avatar corresponding to position of the probe relative to the anatomy; and
display a first image of the anatomy captured by the probe in a second area of the display screen, the anatomy in the first image corresponding to the anatomy icon within the field of view.

2. The system of claim 1, wherein the imaging system is an ultrasound imaging system, and the probe is an ultrasound probe.

3. The system of claim 1, wherein the anatomy includes a heart.

4. The system of claim 1, wherein the processor is configured to display the first image at a first orientation or a second orientation that is different than the first orientation without the probe being moved.

5. The system of claim 1, wherein the processor is further configured to:
display a second image of the anatomy in a third area of the display screen, the second image shows a different area of the anatomy than the first image.

6. The system of claim 5, wherein the second image is captured by the probe before or after the probe captures the first image; and
wherein the processor is configured to display another field of view icon on the display corresponding to the second image.

7. A system configured to assist a user with navigating an instrument relative to a subject, the system comprising:
an imaging system configured to image an anatomy, the imaging system including a probe maneuverable by the user;
a tracking system including a localizer, a probe tracker configured to be mounted to the probe, and a subject tracker configured to be mounted to the subject;
a display device including a display screen; and
a processor configured to:
display a first anatomy icon of the anatomy in a first area of the display screen; and
distinguish visually on the first anatomy icon a first portion of the anatomy that has been imaged by the probe and a second portion of the anatomy that has not been imaged by the probe.

8. The system of claim 7, wherein the processor is further configured to display a second anatomy icon of the anatomy in a second area of the display screen, the second anatomy icon shows a different angle of the anatomy as compared to the first anatomy icon.

9. The system of claim 8, wherein the processor is further configured to display a third anatomy icon of the anatomy in a third area of the display screen, and display a fourth anatomy icon of the anatomy in a fourth area of the display screen;
wherein each of the first anatomy icon, the second anatomy icon, the third anatomy icon, and the fourth anatomy icon are orientated at different angles; and
wherein the processor is configured to distinguish visually on each of the second anatomy icon, the third anatomy icon, and the fourth anatomy icon the first portion of the anatomy that has been imaged by the probe and the second portion of the anatomy that has not been imaged by the probe.

10. The system of claim 7, wherein the anatomy is a heart.

11. A system configured to assist a user with navigating an instrument relative to a subject, the system comprising:
a tracking system including a localizer and a subject tracker configured to be mounted to the subject;
a display device including a display screen; and
a processor configured to:
generate an avatar of the subject and display the avatar on the display screen;
display an anatomy icon of an anatomy on the avatar; and
display a localizer icon and a range icon of the localizer on the display screen at a position relative to the avatar corresponding to position of the localizer relative to the subject.

12. The system of claim 11, wherein the localizer is an electromagnetic localizer.

13. The system of claim 12, wherein the electromagnetic localizer is suspended over an anterior side of the subject.

14. The system of claim 12, wherein the electromagnetic localizer is at a posterior side of the subject.

15. The system of claim 12, wherein the electromagnetic localizer is mounted at a side of the subject.

16. A system configured to assist a user with navigating an instrument relative to a subject, the system comprising:
a respiratory monitoring system including a first respiration sensor and a second respiration sensor;
a display device including a display screen; and
a processor configured to:
generate an avatar of the subject and display the avatar on the display screen;
display a first respiration sensor icon on the avatar at a first location corresponding to placement of the first respiration sensor on the subject; and
display a second respiration sensor icon on the avatar corresponding to placement of the second respiration sensor on the subject.

17. The system of claim 16, wherein the first respiration sensor and the second respiration sensor include electromagnetic localizers.

18. The system of claim 16, wherein the processor is further configured to display on the display screen a respiration meter including a first meter icon and a second meter icon.

19. The system of claim 18, wherein the processor is configured to move the first meter and the second meter icon on the display screen in a manner corresponding to movement of the first respiration sensor and the second respiration sensor during respiration.
